(19) United States
(12) Patent Application Publication
(10) Pub. No.: US 2004/0235767 A1
(43) Pub. Date: Nov. 25, 2004

(54) Anti-angiogenic and immunotherapeutic compositions for cancer treatment

(76) Inventors: Geoffrey Wayne Krissansen, Auckland (NZ); Xueying Sun, Jinan (CN)

Correspondence Address: FISH & RICHARDSON PC, 225 FRANKLIN ST, BOSTON, MA, 02110, US

(21) Appl. No.: 10/478,553

(22) PCT Filed: May 23, 2002
(86) PCT No.: PCT/NZ02/00102

(51) Int. Cl. A61K 48/00

(52) U.S. Cl. 514/44

(57) ABSTRACT

The present invention concerns a method of treating cancer comprising administering to a mammal with cancer an immunotherapeutic agent such as a CAM or a mammalian expression vector containing DNA which encodes a T-cell co-stimulatory CAM, consecutively or sequentially with an anti-angiogenic agent.

### TECHNICAL FIELD OF THE INVENTION

This invention is directed to cancer immunotherapy. In particular the invention is directed at a combination including a CAM-mediated immunotherapy and an anti-angiogenic therapy, which elicits beneficial anti-tumour activity.

### BACKGROUND TO THE INVENTION

Tumours must establish an adequate vascular network to acquire the nutrition necessary for growth and metastasis. Thus, avascular tumours which don't access a blood supply rarely grow beyond a few mm³. The latter notions provide the rationale for anti-angiogenesis therapy of cancers, where the aim is to target the tumour blood supply.¹ Folkman and his colleagues first identified the anti-angiogenic factor angiostatin, representing a fragment of plasminogen.² Angiostatin is a 38,000-Mr protein comprising the first four of five highly homologous 80-amino acid residue long triple-loop structures termed kringles.² It can efficiently inhibit the growth of a broad array of murine and human tumours established in mice,³ and is non-toxic such that tumours can be subjected to repeated treatment cycles.⁴ Its tumour-suppressor activity may arise from its ability to inhibit the proliferation of endothelial cells by binding to the α/β-subunits of ATP synthase⁵, by inducing apoptotic cell death⁶, by subverting adhesion plaque formation and thereby inhibiting the migration and tube formation of endothelial cells,⁷ and/or by down-regulating vascular endothelial growth factor (VEGF) expression.^{8,9} Angiostatin reduces the phosphorylation of the mitogen-activated protein kinases ERK-1 and ERK-2 in human dermal microvascular cells in response to VEGF.¹⁰ Endothelial progenitor cells are exquisitively sensitive to the effects of angiostatin, and may be the most important target of angiostatin.¹¹

Anti-angiogenic therapy with angiostatin requires prolonged administration of recombinant protein in vivo, which is problematical as the production and purification of angiostatin has proven to be difficult. In an alternative approach, gene therapy with viral vectors encoding angiostatin was shown to be effective in preventing the formation of tumour blood vessels, leading to increased tumour cell apoptosis, and decreased tumour growth and metastasis.^{12, 13} However viral vector-based therapies in humans are complicated by issues of safety. Regressed tumours treated with angiostatin regrow when therapy is suspended, but nevertheless, prolonged tumour dormancy can be achieved by several rounds of therapy.^{3, 6} Ultimately dormant tumours could “reawaken”, and hence it would be preferable if angiostatin therapy could be combined with other treatment modalities that would eradicate tumors altogether. For instance, angiostatin has proven to be particularly effective when used in combination with ionizing radiation.^{14,15}

Our previous studies have revealed that optimized gene transfer of several cell adhesion molecules (CAMs) that are able to costimulate T cell proliferation, can cause the complete rejection of small (0.1-0.2 cm in diameter) lymphomas established in mice.¹⁶ CAMs are a general class of molecules that appear to be involved in the initiation and/or aiding of an immune response to tumours. CAMs may include the B7-1 family; and integrin ligands including VCAM-1, MAdCAM-1, and ICAM-1. CAM-mediated immunotherapy is problematical in that it is ineffective against large tumours (>0.3-0.4 cm in diameter), and generates weak anti-tumour systemic immunity.¹⁶ Thus there is a limitation on potential therapeutic use for such compounds as the effect of such compounds is dependent to a significant extent on the size of the tumour. In searching for ways to more effectively harness and strengthen the anti-tumour activity of CAM-mediated immunotherapy, we reported that immunogene therapy employing the T cell costimulator B7-1 could be vastly improved by combining it with the anti-vascular agent 5,6-dimethylxanthenone-4-acetic acid (DMXAA) and/or anti-sense hypoxia-inducible-factor 1α (HIF-1α)^{17, 18}. DMXAA is an agent that has a number of properties including stimulation of inate immunity which anti-angiogenic agents are not currently understood to do. Combination therapy caused the complete and rapid eradication of large tumour burdens (0.4-1.0 cm in diameter) in mice.

### OBJECT OF THE INVENTION

It is an object of the present invention to provide the public with an alternative cancer therapy.

### SUMMARY OF THE INVENTION

According to one aspect of the present invention there is provided a method of treating cancer comprising administering to a mammal with cancer an effective amount of an immunotherapeutic agent comprising a cell adhesion molecule (CAM) or a mammalian expression vector containing DNA which encodes a CAM consecutively or sequentially with an effective amount of an anti-angiogenic agent.

Preferably the effective amounts of the immunotherapeutic agent and the anti-angiogenic agent are synergistically effective amounts.

Preferably, the cancer may be resistant or partially resistant to immunotherapy alone, and more particularly to B7-1-mediated immunotherapy.

Preferably, the cancer may involve advanced or large tumour burdens.

Preferably, the immunotherapeutic agent may comprise an agent capable of inducing B7-1-mediated immunotherapy, and may specifically comprise a B7-1 expression plasmid.

Preferably, the anti-angiogenic agent may include angiostatin gene therapy and/or the gene product of the angiostatin gene.

In a preferred form, the immunotherapeutic agent may be administered prior to the anti-angiogenic agent.

According to a further aspect of the present invention there is provided a method of potentiating the anti-cancer benefits of anti-angiogenic therapy by administering a synergistically effective amount of an immunotherapeutic agent consecutively or sequentially with an effective amount of the anti-angiogenic agent.

According to a further aspect of the present invention there is provided a method of potentiating the anti-cancer activity of angiostatin gene therapy by pre-treatment with a synergistically effective amount of CAM-mediated immunotherapy.

Preferably, the CAM-mediated immunotherapy may include B7-1-mediated immunotherapy.

According to a further aspect of the present invention there is provided the use of an immunotherapeutic agent in the manufacture of a medicament for potentiating the activity of an anti-angiogenic agent against cancer.

According to a further aspect of the present invention there is provided the use of an anti-angiogenic agent in the manufacture of a medicament for potentiating the activity of an immunotherapeutic agent or preferably a CAM-mediated immunotherapeutic agent against cancer.

According to a further aspect of the present invention there is provided the use of an anti-angiogenic agent in the manufacture of a medicament for treating cancer resistant or partially resistant to immunotherapy.

Preferably, the cancer may be resistant or partially resistant to B7-1-mediated immunotherapy.

According to a further aspect of the present invention there is provided the use of an anti-angiogenic agent in the manufacture of a medicament for the treatment of large or resistant tumours.

Preferably, the anti-angiogenic agent may be angiostatin gene therapy and/or a gene product of the angiostatin gene.

Preferably, the immunotherapeutic agent may be an agent capable of facilitating or inducing B7-1-mediated immunotherapy.

According to a further aspect of the present invention there is provided a chemotherapeutic pack including an anti-angiogenic agent and a CAM or a mammalian expression vector containing DNA which encodes a CAM.

Other aspects of the present invention may become apparent from the following description which is given by way of example only and with reference to the specific experimental data.

### DESCRIPTION OF THE DRAWINGS

While the invention is broadly defined above, those persons skilled in the art will appreciate that it is not limited thereto and that it also includes embodiments of which the following description provides examples. In addition, the present invention will be better understood with reference to the accompanying drawings.

FIG. 1. Gene transfer of angiostatin into solid EL-4 tumours in situ impairs tumour development A: Construction of an expression plasmid encoding mouse angiostatin. Complementary DNA encoding the signal sequence (SS), preactivation peptide (PA), and kringles 1-4 of mouse angiostatin was amplified by PCR, and inserted into a pCDNA3 vector, containing the CMV promoter. B, C: Angiostatin expression is enhanced following gene transfer. Subcutaneous EL-4 tumours, 0.4 cm in diameter, were injected with either empty vector (B), or vector encoding angiostatin (C). Illustrated are representative tumour sections prepared 2 days following gene transfer. Angiostatin is stained brown with a mAb recognizing kringles 1-3 of plasminogen. Magnification, ×100. D, E, F, G, H, I: intratumoral gene transfer of angiostatin suppresses tumour angiogenesis independently of VEGF, and induces tumour cell apoptosis. Illustrated are sections prepared from 0.4 cm tumours injected 4 days earlier with either empty pCDNA3 vector (D, F, and H), or vector encoding angiostatin (E, G, and I). They were stained with anti-CD31 (D and E), and anti-VEGF (F and G) mAbs, and by TUNEL analysis for apoptotic cells (H and I; green fluorescent cells showing condensed fragmented nuclei). Magnification, ×100 (F, G, H, and I), ×40 (D and E). J, K: Western blot analysis of intratumoral expression of angiostatin and VEGF following gene transfer. Homogenates of tumour cells extracted from tumours 2 days following gene transfer of either empty plasmid (lanes 1), or angiostatin plasmid (lanes 2) were blotted with an anti-plasminogen mAb recognizing kringles 1-3 (J), and an anti-VEGF mAb (K).

FIG. 2. Gene transfer of angiostatin inhibits the growth of small EL-4 tumours, and synergizes with B7-1 to eradicate large tumours. A: B7-1 immunotherapy eradicates small tumours, whereas angiostatin gene transfer inhibits tumour growth. Established EL-4 tumours, approximately 0.1 cm in diameter, were injected at day 0 with expression plasmids encoding B7-1, angiostatin, or empty vector alone. A repeat injection of angiostatin was administered 48 h later. B: Combining angiostatin gene transfer with B7-1 immunotherapy causes the rapid rejection of large tumours refractory to treatment by B7-1 immunotherapy. Tumours 0.4 cm in diameter were injected with a B7-1 expression plasmid, followed by two injections of angiostatin-encoding plasmid administered 48 h and 96 later. Control tumours were injected with either B7-1 or angiostatin expression plasmid alone, or empty vector. Cured mice were rechallenged by subcutaneous injection of 1×10⁶ EL-4 cells, and followed by another injection of 1×10⁷ EL-4 cells two weeks later, but developed no tumours during the 2 months they were monitored (data not shown). C: The therapeutic efficacy of angiostatin monotherapy against large EL-4 tumours is not improved by increasing gene dosage. Tumours ˜0.4 cm diameter (large tumours) were injected with 100, 150, 200, and 250 μg of angiostatin expression plasmid, as indicated. Each point represents the mean ±SD of results from 5 or 6 mice. D: Combination therapy slows the growth of very large tumours. Tumours 0.6 cm in diameter were either treated as in (B), or with an increased dosage (250 μg) of both B7-1 and angiostatin expression plasmid. Combination therapy with either 100 or 250 μg of each vector completely blocked tumour growth for one week, but then tumours began to regrow. Nevertheless combination therapy was more effective than B7-1 monotherapy. The sizes (diameter in cm) of all tumours were recorded following gene transfer. Complete tumour regression is denoted by vertical arrows. Mice were euthanased when tumours reached more than 1 cm in diameter (denoted by stars).

FIG. 3 Measurement of tumour blood vessel density and apoptosis following gene transfer of angiostatin. A: Tumour blood vessels in sections from control and angiostatin-treated tumours were stained with the anti-CD31 mAb, and counted in blindly chosen random fields to record mean vessel density per surface area (0.155 mm²). n, number of tumours assessed. A significant difference in mean vessel counts between tumours treated with angiostatin versus pcDNA3 (P<0.05) is donated by stars. B: Histograms showing the median and 90th centile distance to the nearest labelling for CD31 from an array point within each tumour. The mean values for tumours treated with pcDNA3 and angiostatin are given (±STD). n, number of tumours assessed. A significant difference in the median or 90th centile distance to nearest blood vessels of tumours treated with pcDNA3 versus (both p<0.01) is donated by stars. C: TUNEL positive cells were counted to record the apoptosis index (AI), which was significantly (p<0.001) different between tumours treated with empty pcDNA3 vector, and vector encoding angiostatin. D: Tumour cells transfected with angiostatin and empty vector control have identical growth rates in vitro. Single cell suspensions prepared from EL-4 tumours 2 days following gene transfer with angiostatin or empty plasmid were seeded at the same concentration, and cultured under identical conditions. The number of viable cells, detected by exclusion of trypan blue, were counted and plotted against d of culture.

### DETAILED DESCRIPTION OF THE INVENTION

The present invention provides a combination therapy for the treatment of large tumours (substantially greater than or equal to 0.4 mm). The therapy is thus effective across a broad range of tumour sizes and is not restricted to smaller less established tumours. The therapy is inevitably restricted to tumours that are dependent on a blood supply, and are susceptible to immunotherapy, albeit possibly only in combination with an anti-angiogenic drug. The present invention has wide utility, since the majority of solid tumours in animals, independent of species, depend on a blood supply in order to survive and grow. While the invention describes results with lymphoma, it is expected to be applicable to most vascular tumours. Further, good research progress is being made in determining how tumors evade the immune response, and hence how they can be rendered susceptible to immunotherapy. The application of the invention to a particular tumour, and the doses of each agent required to cause tumour rejection or delay tumour growth will depend on the nature of the tumour, irrespective of the tumour type or host. Thus, increased doses of anti-angiogenic reagents, or a combination of such reagents, will need to be applied to tumours that secrete large amounts of competing pro-angiogenic factors. Likewise, one or more immunotherapeutic agents, may be required to combat tumours that are non-immunogenic, and/or immunosuppressive. Ultimately, these parameters will need to be determined for each individual tumour. Monotherapies can only give an indication of treatment possibilities, and ultimately a combination of an immunotherapeutic agent with an anti-angiogenic agent should be applied.

Anti-angiogenic therapy may include an angiostatin gene therapy. The angiostatin gene therapy is preferred because of the lease of construction of an angiostatin expression vector. Gene transfer of therapeutic proteins overcomes the problem of having to source, and produce sufficient quantities of protein for therapy. This is important in the case of anti-angiogeneic factors such as angiostatin that are short-lived proteins,² and have to be administered continuously in large amounts for optimum and sustained benefit.²⁶

Gene transfer of angiostatin into tumours leads to the continuous secretion of angiostatin by the tumours themselves. However, the applicant has found that results obtained with angiostatin monotherapy were not as spectacular as those previously obtained using recombinant angiostatin protein,²⁶ or viral vectors encoding angiostatin¹².

Anti-angiogenic proteins are effective at inducing tumour regression, but are not directly tumoricidal, and hence tumour regrowth frequently occurs once treatment is suspended. The effect is one of competition within pro-angiogenic factors. The applicant herein describes for the first time that anti-angiogenic therapy (eg angiostatin gene therapy) and immunotherapy (eg CAM-mediated immunotherapy, and preferably B7-1 immunotherapy) produce a synergistic effect in causing a rapid and substantially complete rejection, or reduced growth, of large EL-4 tumours. In contrast, B7-1 monotherapy was ineffective against large tumours, despite the fact that the tumours naturally expressed low levels of endogenous angiostatin. The latter can presumably be attributed to the local production of proangiogenic factors by the tumour, such as VEGF that was readily detectable, which outweigh the effects of endogenously produced angiostatin. The combination therapy disclosed herein therefore extends the effective range of B7-1 therapy.

It will be appreciated that CAM-mediated immunotherapy is not limited to B7-1-mediated immunotherapy, and that the use of other CAM-mediated immunotherapy in this combination therapy, such as B7-2 or an integrin ligand (including VCAM-1, MAdCAM-1 and ICAM-1) or a mammalian expression vector containing DNA which encodes a CAM, or another immunotherapeutic agent capable of stimulating anti-tumour immunity, does not depart from the scope or spirit of the invention.

Surprisingly, mice cured by combination therapy, as herein described, and rechallenged with live EL-4 cells remained tumour-free for at least 2 months, indicating that potent systemic anti-tumour immunity had been generated.

In a preferred embodiment the delivery of the reagents is timed such that the B7-1 expressed vectors are administered first, followed by angiostatin. Angiostatin may facilitate B7-1 immunotherapy by inhibiting tumour cell growth, thereby preventing the outgrowth of escape variants; and by rendering the tumour accessible to immune cells as the tumour begins to disintegrate. In this regard, the AI (Apoptosis Index) of EL-4 tumours treated with angiostatin monotherapy was greater than that of control tumours treated with empty vectors, as described previously.^{3,8} Angiostatin causes endothelial cell apoptosis,^{7,31} and as shown here inhibits neovascularization, potentially restricting the supply of tumour cell survival factors provided either by endothelial cells or by the circulation.

The effects of angiostatin may be mediated, in part, by the ability of angiostatin to downregulate VEGF. VEGF is a potent endothelial mitogen and permeability factor, commonly expressed in tumours, which has been implicated in pathologic angiogenesis.³² It appears to play a critical role in tumour formation, as tumour growth can be inhibited by antibody-mediated blockade of VEGF,³³ and by administration of antisense VEGF mRNAs³⁴, or retrovirus encoding a soluble mutant form of the VEGF receptor.³⁵ Tumour angiogenesis is a multistep process, where the switch to the angiogenic phenotype requires both upregulation of angiogenic stimulators and downregulation of angiogenesis inhibitors.^{36,37} Thus, overproduction of angiogenic stimulators such as VEGF is necessary, but not sufficient for tumour vascularization.

It is herein shown by the applicant that exogenous angiostatin inhibited the formation of small, potentially nascent tumour blood vessels. The anti-vascular activity of angiostatin appeared to be independent of effects on VEGF expression. The applicant has found that angiostatin therapy led to a slight increase in intratumoral VEGF expression. Ding et al recently reported that intratumoral administration of endostatin caused a compensatory increase in situ of VEGF and VEGF receptor mRNA expression.³⁸ The applicant predicts that tumours may antagonize anti-angiogenic therapy by overproducing angiogenic factors to overcome the imposed increasing hypoxia. Angiostatin may vary in its effects on VEGF expression depending on the origin of the angiostatin molecule administered, and the type and species origin of the tumour. Thus, administration of angiostatin, purified from human plasma, inhibited VEGF expression in rat C6 and 9L gliomas, but not in human U-87 gliomas.⁸ In contrast, administration of recombinant E.coli-derived angiostatin (kringles 1-3) almost completely prevented VEGF expression in U-87 tumours.⁹

The invention is now described by specific reference to the following experimental section.

Experimental

Methods and Materials

Mice and Cell Lines

Male C57BL/6 mice (H-2b), 6-8 weeks old, were obtained from the Animal Resource Unit, School of Medicine and Health Science, University of Auckland, Auckland, New Zealand. The syngeneic (H-2b) EL-4 thymic lymphoma cell line was purchased from the American Type Culture Collection (Rockville, Md., USA). It was cultured at 37° C. in DMEM medium (Gibco BRL, Grand Island, N.Y., USA), supplemented with 10% foetal calf serum, 50 U/mL penicillin/streptomycin, 2 mM L-glutamine, and 1 mM pyruvate.

Plasmid Construction

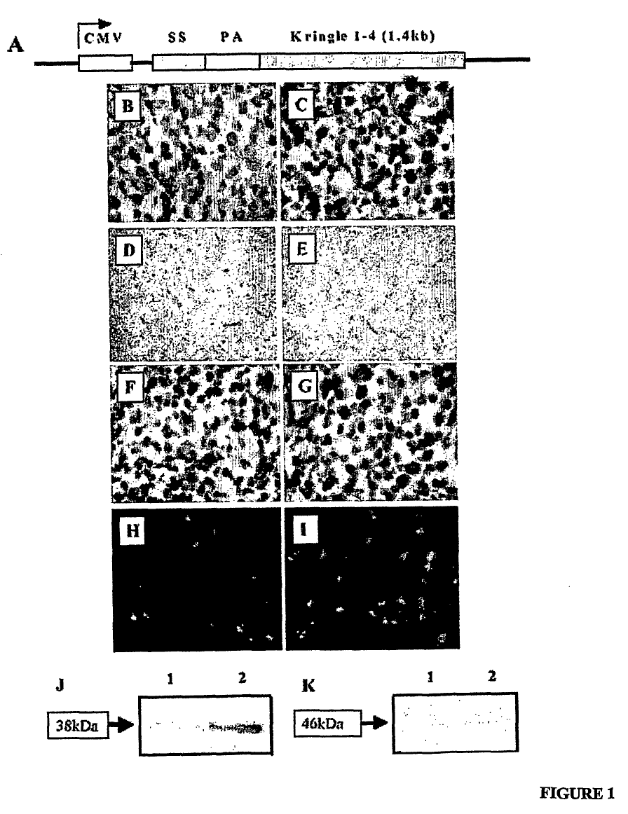

Complementary DNA encoding the signal peptide and first four kringle regions of mouse plasminogen was PCR amplified from IMAGE clone 1920527 with oligonucleotide primers 5′-ACGAAGCTTGGATCCATGGACCATAAGGAAGTA-3′ and 5′-ACGTCTAGACTCGAGTTATGTGGGCAATTCCACAACA-3′, corresponding to amino acid residues 1-6 and 461-466, respectively, as described previously.¹⁹ The 1.4-kb mouse angiostatin PCR product was subcloned into the pcDNA3 expression plasmid (Invitrogen) (FIG. 1A). The pCDM8 expression plasmid encoding mouse B7-1 was constructed using a full-length 1.2 kb B7-1 cDNA clone (kindly provided by Dr P Linsley, Bristol Myers Squibb, Seattle, Wash.). The integrity of all cDNA constructs was confirmed by DNA sequence analysis.

Gene Transfer of Expression Plasmids and Monitoring Anti-Tumour Activity

Purified plasmids were diluted in a solution of 5% glucose in 0.01% Triton X-100, and mixed in a ratio of 1:3 (wt:wt) to a final concentration of 1 mg/mL with DOTAP cationic liposomes (Boehringer Mannheim, Mannheim, Germany). As described previously the latter is a particularly efficient transfection vehicle.¹⁶ Tumours were established by injection of 2×10⁵ EL-4 tumour cells into the right flank of mice, and tumour growth determined by measuring two perpendicular diameters. Animals were killed when tumours reached more than 1 cm in diameter, in accord with Animal Ethics Committee approval (University of Auckland). Tumours reached 0.1 to 0.4 cm in diameter in 14 to 18 days, and were injected with 100 μl of expression plasmids (100 μg). For combinational treatment, reagents were delivered in a timed fashion, as described previously¹⁷. Thus, B7-1 cDNA was injected first, followed by two injections of angiostatin cDNA 48 and 96 h subsequent to B7-1 gene transfer. Control tumours were injected with empty vector. Cured mice were rechallenged 3 weeks after tumour disappearance by injecting 1×10⁶ EL-4 cells subcutaneously into the opposing flank (left flank), followed by an even larger challenge of 1×10⁷ EL-4 cells two weeks later. All experiments included 6 mice per treatment group, and each experiment was repeated at least once.

Immunohistochemistry

Tumour cryosections (10 μm) prepared 2 days following gene transfer underwent overnight incubation with either an anti-B7-1 mAb (1G10, Pharmingen, San Diego, Calif.), an anti-plasminogen mAb recognizing kringles 1-3 (Calbiochem-Novabiochem Corp., CA), or a rabbit polyclonal antibody against VEGF (Ab-1, Lab Vision Corp., CA). They were subsequently incubated for 30 min with appropriate secondary antibodies (VECTASTAIN Universal Quick kit, Vector Laboratories, Burlingame, Calif.), and developed with Sigma FAST DAB (3,3′-diaminobenzidine tetrahydrochloride) and CoCl₂ enhancer tablets (Sigma). Sections were counterstained with Mayer's hematoxylin.

Assessment of Vascularity

The methodology to determine tumour vascularity has been described previously²⁰. Briefly, 10 μm frozen tumour sections prepared 4 days after gene transfer were immunostained with the anti-CD31 antibody MEC13.3 (Pharmingen, CA), as described above. Stained blood vessels were counted in five blindly chosen random fields (0.155 mm²) at 40×magnification, and the mean of the highest three counts was calculated. The concentric circles method^{21,22} was used to assess vascularity.

In situ Detection of Apoptotic Cells

Serial sections of 6 μm thickness were prepared from excised tumours that had been frozen in liquid nitrogen, and stored at −70° C. TUNEL staining of sections was performed using an in situ apoptosis detection kit from Boehringer Mannheim, Germany. Briefly, frozen sections were fixed with 4% paraformaldehyde solution, permeabilized with a solution of 0.1% Triton-X100 and 0.1% sodium citrate, incubated with TUNEL reagent for 60 min at 37° C., and examined by fluorescence microscopy. Adjacent sections were counterstained with haematoxylin and eosin. The total number of apoptotic cells in 10 randomly selected fields were counted. The apoptotic index was calculated as the percentage of positive staining cells, namely AI=number of apoptotic cells×100/total number of nucleated cells.

Cell Proliferation Assay

To determine whether gene transfer of angiostatin affected the growth of EL-4 cells in vitro, single cell suspensions were prepared from EL-4 tumours following gene transfer of either angiostatin or empty plasmid, and seeded at 1×10⁴ cells/well on 24-well culture plates in triplicate. Cells were grown at 37° C. in DMEM medium (Gibco BRL, Grand Island, N.Y., USA), supplemented with 10% foetal calf serum, 50 U/mL penicillin/streptomycin, 2 mM L-glutamine, and 1 mM pyruvate. Cells were enumerated at 24-h intervals with a hemacytometer.

Western Blotting

Tumours injected 2 days earlier with angiostatin or empty expression plasmids were excised, minced with scissors and homogenized in protein lysate buffer (50 mmol/L Tris pH 7.4, 100 μmol/L EDTA, 0.25 mol/L sucrose, 1% SDS, 1% NP40, 1 μg/ml leupeptin, 1 μg/ml pepstatin A and 100 μmol/L phenylmethylsulfonyl fluoride) at 4° C. using a motor driven Virus homogenizer (Virtus, USA). Tumour lysates from each group of mice were pooled, and protein content determined. Tissue or cell debris was removed by centrifugation at 10,000×g for 10 min at 4° C. Protein samples (100 μg) were resolved on 10% polyacrylamide SDS gels under reducing conditions, and electrophoretically transferred to nitrocellulose Hybond C extra membranes (Amersham Life Science, England). After blocking the membranes with 5% BSA in TTBS (20 mmol/L Tris, 137 mmol/L NaCl pH 7.6 containing 0.1% Tween-20), blots were incubated with the anti-plasminogen mAb, or the Ab-1 rabbit polyclonal antibody against VEGF. They were then incubated with horseradish peroxidase-conjugated secondary antibodies, and developed by enhanced chemiluminescence (Amersham International plc, England) and exposure to X-Ray film. Band density was quantified using Scion Image software.

Statistical Analysis

Results were expressed as mean values±standard deviation (SD). A student's T test was used for evaluating statistical significance, where a value less than 0.05 (p<0.05) denotes statistical significance.

Results

Gene Transfer of an Angiostatin Expression Plasmid into Tumours in situ Weakly Retards Tumour Growth

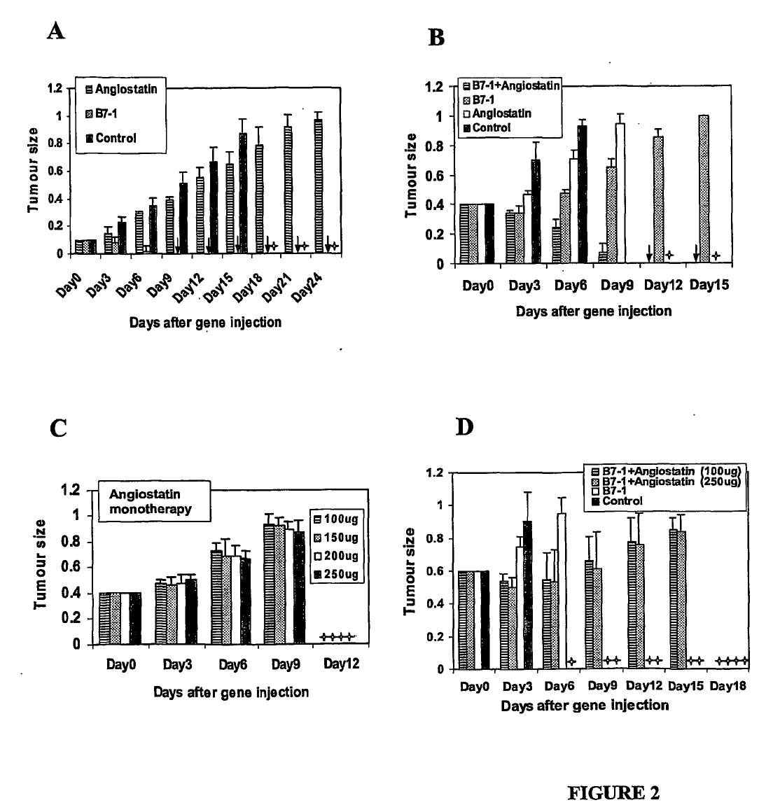

Small EL-4 tumours (0.1 cm in diameter) were injected with a DNA/liposome transfection vehicle containing 100 μg of angiostatin (FIG. 1A), B7-1 expression plasmid DNA, or control empty vector. Tumours grew rapidly in the control group, reaching 1 cm in size 14-17 days following gene transfer, whereas those treated with B7-1 plasmid completely and rapidly regressed within one week of gene transfer (FIG. 2A), as described previously.¹⁶⁻¹⁸ In contrast, tumours treated with angiostatin plasmids were weakly inhibited in their growth, reaching 1 cm in size 22-26 days following gene transfer. A repeat injection of angiostatin plasmid 48 hours after the first injection increased the therapeutic efficacy, but still failed to cause sustained tumour regression (data not shown). Failure of angiostatin to cause significant tumour regression was not due to poor expression of the plasmid, as immunohistochemical staining of tumour sections, prepared 2 days following gene transfer, with an antibody recognizing kringles 1-3 of plasminogen, revealed intense staining indicating that angiostatin was overexpressed in tumours (FIG. 1C). In contrast, sections prepared from control tumours treated with 160 μg of empty vector revealed only weak endogenous levels of angiostatin (FIG. 1B). Western blotting of homogenates of tumour cells extracted from tumours confirmed that angiostatin was over-expressed (3-fold) in transfected tumours, and that it was produced at the expected size of 38 kDa (FIG. 1J).

Vascular Attack by Angiostatin Synergizes with B7-1 Immunotherapy to Eradicate Large Tumours

We previously reported that an attack on tumour blood vessels by either the anti-angiogenic drug DMXAA,¹⁷ or an expression vector encoding antisense HIF-1α in combination with B7-1-mediated immunotherapy, overcomes tumour immune-resistance and leads to the eradication of large tumours. As reported previously,^{17,18} gene transfer of a B7-1 expression plasmid into large tumours (0.4 cm in diameter) halved the rate of tumour growth, but was not able to induce sustained regression of EL-4 tumours. This was confirmed with B7-1 alone in the present study (FIG. 2B). The failure of B7-1 therapy was not the result of poor transfection efficiency as B7-1 gene transfer led to expression of B7-1 in 90% of tumour cells (data not shown), in accord with our previous results.^{16,18}

The treatment of tumours 0.4 cm in diameter with angiostatin gene therapy only weakly inhibited tumour growth (FIG. 2B). Therapeutic efficacy against large tumours was not improved by increasing the gene dosage of angiostatin up to 250 μg (FIG. 2C).

For combination therapy, tumours 0.4 cm in diameter were first injected with DNA/liposome complexes containing 100 μg B7-1, followed by two injections of angiostatin expression plasmid 48 and 96 h later. Combined gene therapy led to complete tumour regression within 10 days, and mice remained tumour-free for 3 weeks (FIG. 2B).

Two injections of angiostatin were applied for combination therapy as only 50% of mice were cured by a single injection. To determine whether systemic anti-tumour immunity had been generated, cured mice were rechallenged in the opposing flank with 1×10⁶ parental tumour cells, and two weeks later by an even larger challenge of 1×10⁷ parental tumour cells. Such mice remained tumour-free for the 2 months they were monitored (data not shown). In a more strenuous test of therapeutic efficacy, advanced tumours 0.6 cm in diameter were treated by combination therapy. Unfortunately, the treatment proved ineffective in eradicating very large tumours even when the gene dosage of both B7-1 and angiostatin was increased to 250 μg, but nevertheless combination treatment retarded tumour growth more effectively than B7-1 monotherapy (FIG. 2D).

Over-Expressed Angiostatin Reduces the Density of Tumour Blood Vessels, and Increases Tumour Cell Apoptosis Independently of VEGF

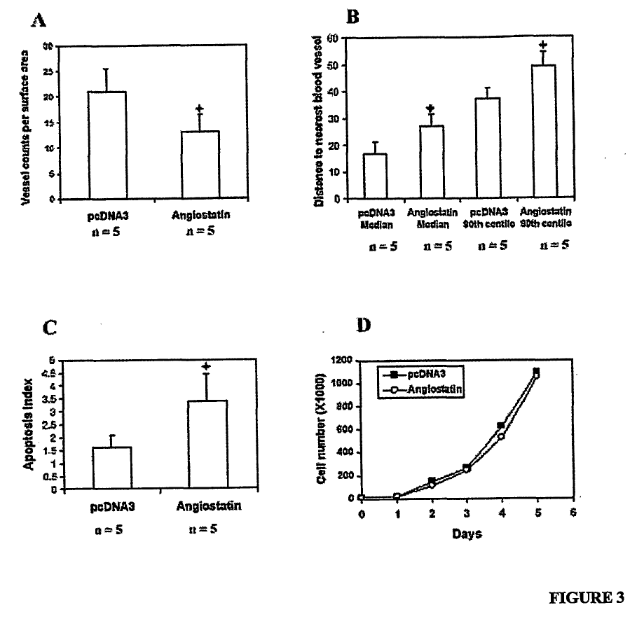

Gene transfer of angiostatin inhibited tumour angiogenesis, as evidenced by a statistically significant (p<0.05) reduction in tumour vessel density (FIGS. 1D and E; and FIG. 3A). The median and 90th centile distances to the nearest CD31-labelled venules from an array of points within tumours treated with angiostatin were significantly (p<0.01) longer than those in tumours treated with empty vector (FIG. 3B). There appeared to be a selective loss of small, presumably nascent vessels, after angiostatin therapy (FIG. 1, compare D and E). The introduced angiostatin had no inhibitory effect on tumoral VEGF expression, as detected by immunohistochemical staining of tumour sections with an anti-VEGF specific antibody (FIGS. 1F and G). Rather VEGF expression was slightly elevated (1.5-fold) as revealed by Western blot analysis of homogenates of tumour cells extracted from tumours (FIG. 1K), presumably due to the increased hypoxic environment resulting from decreased blood vessel density.

The rate of tumour cell apoptosis measured by in situ labelling of fragmented DNA with terminal transferase (TUNEL method) was significantly (p<0.001) increased in angiostatin-treated tumours compared to control tumours (FIGS. 1H and I; FIG. 3C). Over-expression of angiostatin had no detectable affect on EL-4 cell growth/viability per se, as there was no significant difference (p>0.05) in the morphological appearance or growth rate of EL-4 transfectants harvested from angiostatin-treated tumours versus those treated with empty plasmid (FIG. 3D).

These results confirm that cancer immunotherapy employing B7-1 or other CAMs alone is of limited therapeutic use against large tumours. However, the therapeutic efficacy of CAM-mediated immunotherapy can be harnessed by combination with other treatment modalities, and in particular—as described herein—a synergistic benefit may be achieved with angiostatin gene therapy, thereby rendering large tumours susceptible to immune attack. The combination therapy of CAM-mediated immunotherapy and angiostatin gene therapy may be used as a treatment for human cancers. Following applicant's surprising determination of the effect of the combination effect as described herein, effective dose rates simply being a matter of trial and error well within the abilities of a skilled person to determine as has been discussed previously herein. If it is decided to start therapy with a mono-therapeutic approach, then the efficacy restriction for monotherapy based on tumour size will also be able to be readily determined by trial and error. Results obtained with monotherapy may have no bearing on the potential utlility of combination therapy, and ultimately combination therapy (the invention) should be trialled.

In general, compounds of this invention will be administered in therapeutically (ie synergistically) effective amounts by any of the usual modes known in the art, either singly or in combination with one other compound of this invention and/or at least one other similar therapeutic agent for the cancer being treated. A therapeutically effective amount may vary widely depending on the cancer, its severity, the age and relative health of the animal being treated, the potency of the compound(s), and other factors. A person of ordinary skill in the art will be able without undue experimentation, having regard to that skill and this disclosure, to determine a therapeutically effective amount of compounds of use in this invention for a given cancer.

As an immunotherapeutic agent, therapeutically effective amounts of the CAM (eg B7-1 expression plasmid or agent) in this invention may range from about 10 μg to 10 mg for intratumoral injection, with higher doses being appropriate for administration by methods such as systemic (eg. transdermal), or parenteral (e.g. intravenous, intramuscular) administration. A person of ordinary skill in the art will be able without undue experimentation, having regard to that skill and this disclosure, to determine a therapeutically effective amount of a compound of this invention for a given disease or injury.

As an anti-angiogenic agent, therapeutically effective amounts of angiostatin expression plasmid or agent in this invention may range from about 10 μg to 10 mg for intratumoral injection, with higher doses being appropriate for administration by methods such as systemic (eg. transdermal), parenteral (e.g. intravenous, intramuscular) administration. A person of ordinary skill in the art will be able without undue experimentation, having regard to that skill and this disclosure, to determine a therapeutically effective amount of a compound of this invention for a given disease or injury.

In general, compounds of this invention will be administered as pharmaceutical compositions by one of the following routes: oral, topical, systemic (eg. transdermal, intranasal, or by suppository), parenteral (eg. intramuscular, subcutaneous, or intravenous injection), intratumoral (eg. by injection, using ballistics); by implantation, and by infusion through such devices as osmotic pumps, transdermal patches, and the like. Compositions may take the form of tablets, pills, capsules, semisolids, powders, sustained release formulation, solutions, suspensions, elixirs, aerosols, injections, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Gennaro A R: Remington: The Science and Practice of Pharmacy, 20^{th} ed., Lippincott, Williams & Wilkins, 2000. Suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also especially suitable for administration of the compound to tumours.

Compounds compatible with this invention might also suitably be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing the compound are prepared by methods known per se: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy.

Compounds of this invention may also be PEGylated to increase their lifetime in vivo, based on, e.g., the conjugate technology described in WO 95/32003.

The amount of a compound of this invention in the composition may vary widely depending on the type of composition, size of a unit dosage, kind of excipients, and other factors well known to those of ordinary skid in the art. The combination of compounds could be provided to a user in a chemotherapeutic pack for ease of use and access. The pack could be constructed in any suitable manner as would be known to the skilled person.

Compounds may be administered as naked DNAs, or using virus technologies, or as recombinant proteins, peptides, or pharmaceutical compositions, or by other means that any person of ordinary skill in the art would be able to devise.

While in the foregoing description there has been made reference to specific components or integers of the invention having known equivalents then such equivalents are herein incorporated as if individually set forth.

Although this invention has been described by way of example only and with reference to possible embodiments thereof it is to be understood that modifications or improvements may be made without departing from the scope or spirit of the invention as defined in the appended claims.

References

1. Oehler M K, Bicknell R. The promise of anti-angiogenic cancer therapy. Brit J Cancer. 2000;82:749-752.

2. O'Reilly M S, Holmgren L, Shing Y, et al. Angiostatin: a novel angiogenesis inhibitor that mediates the suppression of metastases by a Lewis lung carcinoma. Cell. 1994;79:315-328.

3. O'Reilly M S, Holmgren L, Chen C, et al. Angiostatin induces and sustains dormancy of human primary tumours in mice. Nat Med. 1996;2:689-692.

4. Boehm T, Folkman J, Browder T, et al. Antiangiogenic therapy of experimental cancer does not induce acquired drug-resistance. Nature 1997;390:404-407.

5. Moser T L, Stack M S, Asplin I, et al. Angiostatin binds ATP synthase on the surface of human endothelial cells. Proc Natl Acad Sci USA 1999;96:2811-2816.

6. Holmgren L, O'Reilly M S, Folkman J. Dormancy of micrometastases-balanced proliferation and apoptosis in the presence of angiogenesis suppression. Nat Med. 1995;1:149-153.

7. Claesson-Welsh L, Welsh M, Ito N, et al. Angiostatin induces endothelial cell apoptosis and activation of focal adhesion kinase independently of the integrin-binding motif RGD. Proc Natl Acad Sci USA. 1998;95:5579-5583.

8. Kirsch M, Strasser J, Allender R, et al. Angiostatin suppresses malignant glioma growth in vivo. Cancer Res. 1998;58:4654-4659.

9. Joe Y A, Hong Y K, Chung D S, et al. Inhibition of human malignant glioma growth in vivo by human recombinant plasminogen kringles 1-3. Int J Cancer 1999;82:694-699.

10. Redlitz A, Daum G, Sage E H. Angiostatin diminishes activation of the mitogen-activated protein kinase ERK-1 and ERK-2 in human dermal microvascular endothelial cells. J Vasc Res. 1999;36:28-34.

11. Ito H, Rovira II, Bloom M L, et al. Endothelial progenitor cells as putative targets for angiostatin. Cancer Res. 1999;59:5875-5877.

12. Tanaka T, Cao Y, Folkman J, et al. Viral vector-targeted antiangiogenic gene therapy utilizing angiostatin complementary DNA. Cancer Res. 1998;58:3362-2269.

13. Griscelli F, Li H, Griscelli A B, et al. Angiostatin gene transfer:inhibition of tumour growth in vivo by blockage of endothelial cell proliferation associated with a mitosis arrest. Proc Natl Acad Sci USA 1998;95:6367-6372.

14. Gorski D H, Mauceri H J, Salloum R M., et al. Potentiation of the anti-tumour effect of ionizing radiation by brief concomitant exposures to angiostatin. Cancer Res. 1998;58:5686-5689.

15. Mauceri H J, Hanna N N, Beckett M A, et al. Combined effects of angiostatin and ionizing radiation in anti-tumour therapy. Nature 1998;394:287-291.

16. Kanwar J R, Berg R W, Lehnert K, et al. Taking lessons from dendritic cells: multiple xenogeneic ligands for leukocyte integrins have the potential to stimulate anti-tumour immunity. Gene Ther. 1999;6:1835-1844.

17. Kanwar J R, Kanwar R K, Pandey S, et al. Vascular attack by 5,6-dimethylxanthenone-4 acetic acid combined with B7-1-mediated immunotherapy overcomes immune-resistance and leads to the eradication of large tumours. Cancer Res. 2000;61:1948-1956.

18. Sun X, Leung E, Kanwar J R, et al. Gene transfer of antisense hypoxia inducible factor-1α enhances the therapeutic efficacy of cancer immunotherapy. Gene Ther. 2001;8:638-645.

19. Cao Y, O'Reilly M S, Marshall B, et al. Expression of angiostatin cDNA in a murine fibrosarcoma suppresses primary tumour growth and produces long-term dormancy of metastases. J Clin Invest. 1998;101:1055-1063.

20. Heather E R, Jessica L, Randall S J. HIF-1α is required for solid tumour formation and embryonic vascularization. EMBO J. 1998;17:3005-3015.

21. Kayar S R, Archer P G, Lechher A J, Banchero N. Evaluation of the concentric-circles method for estimating capillary-tissue diffusion distances. Microvascular Res. 1982;24:342-353.

22. Maxwell P H, Dachs G U, Gleadle J M, et al. Hypoxia-inducible factor-1 modulates gene expression in solid tumors and influences both angiogenesis and tumor growth. Proc Natl Acad Sci USA. 1997;94:8104-8109.

23. Baguley B C, Ching L M. Immunomodulatory actions of xanthenone anticancer agents. BioDrugs 1997;8:119-127.

24. Pedley R B, Boden J A, Boden R, et al. Ablation of colorectal xenografts with combined radioimmunotherapy and tumor blood flow-modifying agents. Cancer Res. 1996;56:3293-3300.

25. Finlay G J, Ching L M, Wilson W R, et al. Resistance of cultured Lewis lung carcinoma cell lines to tiazofurin. J Nat Cancer Inst. 1987;79:291-296.

26. Drixler T A, Rinkes I H, Ritchie E D, et al. Continuous administration of angiostatin inhibits accelerated growth of colorectal liver metastases after partial hepatectomy. Cancer Res. 2000;60:1761-1765.

27. Chen Q-R, Kumar D, Stass S A, et al. Liposomes complexed to plasmids encoding angiostatin and endostatin inhibit breast cancer in nude mice. Cancer Res. 1999;59:3308-3312.

28. Sacco M G, Caniatti M, Cato E M, et al. Liposome-delivered angiostatin strongly inhibits tumor growth and metastatization in a transgenic model of spontaneous breast cancer. Cancer Res. 2000;60:2660-2665.

29. Ambs S, Dennis S, Fairman J, et al. Inhibition of tumor growth correlates with the expression level of a human angiostatin transgene in transfected B16F10 melanoma cells. Cancer Res. 1999;59:5773-5777.

30. Blezinger P, Wang J, Gondo M. et al. Systemic inhibition of tumour growth and tumour metastases by intramuscular administration of the endostatin gene. Nature Biotech. 1999;17:343-348.

31. Lucas R, Holmgren L, Garcia I, Jimenez B, Mandriota S J, Borlat F, Sim B K L, Wu Z, Grau G E, Shing Y, Soff G A, Bouck N, and Pepper M S. Multiple forms of angiostatin induce apoptosis in endothelial cells. Blood. 1998;92:4730-4741.

32. Ferrara N, Houk L, Jakeman L, et al. Molecular and biological properties of the vascular endothelial cell growth factor family of proteins. Endocr Rev. 1992;13:18-32.

33. Borgstrom P, Killan K J, Sriramarao P, et al. Complete inhibition of angiogenesis and growth of microtumours by anti-vascular endothelial growth factor neutralizing antibody: novel concepts of angiostatic therapy from intravital video microscopy. Cancer Res. 1996;56:4032-4039.

34. Saleh M, Stacker S A, Wilks A F. Inhibition of growth of C6 glioma cells in vivo by expression of antisense vascular endothelial growth factor sequence. Cancer Res. 1996;56:393-401.

35. Millaruer B, Shawver L K, Plate K H, et al. Glioblastoma growth inhibited in vivo by a dominant-negative Flk-1 mutant. Nature 1994;367:576-579.

36. Hanahan D, Folkman J. Patterns and emerging mechanisms of the angiogenic switch during tumorigenesis. Cell 1996;86:353-364.

37. Folkman J. Angiogenesis in cancer, vascular, rheumatoid and other disease. Nature Med. 1995;1:27-31.

38. Ding I, Sun J Z, Fenton B, Liu W M, Kimsely P, Okunieff P, and Min W. Intratumoral administration of endostatin plasmid inhibits vascular growth and perfusion in Mca-4 mammary carcinomas. Cancer Res. 2001;61:526-531.

1. A method of treating cancer comprising administering to a mammal with cancer an effective amount of immunotherapeutic agent comprising a cell adhesion molecule (CAM) or a mammalian expression vector containing DNA which encodes a CAM consecutively or sequentially with an effective amount of anti-angiogenic agent.

2. A method according to claim 1 wherein the amounts of immunotherapeutic agent and anti-angiogenic agent are synergistically effective amounts.

3. A method according to claim 1 wherein the cancer is resistant or partially resistant to immunotherapy alone.

4. A method according to claim 3 wherein the cancer is resistant or partially resistant to B7-1 mediated immunotherapy.

5. A method according to claim 1 wherein the cancer involves advanced or large tumour burdens.

6. A method according to claim 1 wherein the immunotherapeutic agent includes an agent capable of inducing B7-1 mediated immunotherapy.

7. A method according to claim 6 wherein the immunotherapeutic agent comprises a B7-1 expression plasmid.

8. A method according to claim 1 any one of the preceding wherein the anti-angiogenic agent includes angiostatin gene therapy and/or the gene product of the angiostatin gene.

9. A method according to claim 1 wherein the immunotherapeutic agent is administered before the anti-angiogenic agent.

10. A method of potentiating the anti-cancer benefits of anti-angiogenic therapy by administering a synergistically effective amount of an immunotherapeutic agent consecutively or sequentially with an effective amount of the anti-angiogenic agent.

11. A method of potentiating the anti-cancer activity of angiostatin gene therapy by pre-treatment with an effective amount of CAM-mediated immunotherapy.

12. A method according to claim 11 wherein the CAM-mediated immunotherapy includes B7-1 mediated immunotherapy.

13-18. Canceled.

19. A chemotherapeuatic pack including an anti-angiogenic agent and an immunotherapeutic agent when used in accordance with a method of claim 1.

20. A chemotherapeutic pack according to claim 19 wherein the anti-angiogenic agent is angiostatin gene therapy and/or the gene product of the angiostatin gene.

21. A chemotherapeutic pack according to claim 19 wherein the immunotherapeutic agent is capable of inducing B7-1 mediated immunotherapy.

22. A method according to claim 2 wherein the cancer is resistant or partially resistant to immunotherapy alone.

23. A method according to claim 2 wherein the cancer involves advanced or large tumour burdens.

24. A method according to claim 2 wherein the immunotherapeutic agent includes an agent capable of inducing B7-1 mediated immunotherapy.

25. A method according to claim 4 wherein the immunotherapeutic agent includes an agent capable of inducing B7-1 mediated immunotherapy.
